(19) United States
(12) Patent Application Publication
(10) Pub. No.: US 2022/0249055 A1
(43) Pub. Date: Aug. 11, 2022

(54) NON-INVASIVE, REAL-TIME, BEAT-TO-BEAT, AMBULATORY BLOOD PRESSURE MONITORING

(71) Applicant: DP Holding (U.K) Limited, Grantham (GB)

(72) Inventors: Darren SPENCER, Grantham (GB); Peter BALMFORTH, Grantham (GB)

(21) Appl. No.: 17/629,742

(22) PCT Filed: Jul. 24, 2020
(86) PCT No.: PCT/GB2020/051784

§ 371 Date: Jan. 24, 2022

(30) Foreign Application Priority Data
Jul. 25, 2019 (GB) 1910660.8
Aug. 6, 2019 (GB) 1911247.3
Aug. 6, 2019 (GB) 1911249.9
Aug. 6, 2019 (GB) 1911251.5

(51) Int. Cl. A61B 8/00; A61B 8/04; A61B 8/08; A61B 8/02

(52) U.S. Cl. CPC A61B 8/4236; A61B 8/4281; A61B 8/4411; A61B 8/04; A61B 8/02; A61B 8/486; A61B 8/565; A61B 8/5223; A61B 8/4488

(57) ABSTRACT

According to an aspect of the invention, there is provided an ambulatory system for determining a cardiac parameter at a fixed location within the cardiovascular system of a subject. The system comprises a wearable sensor including an ultrasound transducer. The wearable sensor can contact the skin of the subject and be positioned proximate to the fixed location. The system comprises a data collection module that is in communication with the ultrasound transducer. The ultrasound transducer is configured to detect a pressure wave passing through the fixed location. The data collection module is configured to collect data relating to the pressure wave passing through the fixed location, analyse the pressure wave, and determine at least one cardiac parameter based on the analysis.

### FIELD

The present invention is in the field of real-time wearable sensor technologies that are used to monitor blood pressure (BP) including peripheral blood pressure. Sensors may include ultrasound sensors. Live data feeds from such real time sensors can either be downloaded and read post recording or can deliver live data feed using Wi-Fi/4G/Bluetooth mobile telecommunications networks to remote devices.

### BACKGROUND

Non-invasive blood pressure monitoring in typically relies upon decades-old sphygmomanometer measurement. According to this approach, an inflatable cuff applied to a limb or extremity is used create a supra-systolic pressure allowing measurement of systolic and diastolic pressure in the limb as the air in the cuff is released. In the doctors' surgery, or with home monitoring, this captures a single moment in time the blood pressure (BP) of the individual in a resting state. However, this measurement does not represent any variability in blood pressure that occurs through the day or night. 24-hour ambulatory BP monitoring can be used to gain a wider snapshot of BP variation throughout the day's activities. Nevertheless, this presents a challenge during the evening and at night as the devices are typically uncomfortable to wear, with the repeated cuff inflation/deflation cycles often waking the subject creating a “false representation” of night time and overall 24-hour blood pressure.

During contraction of the heart, a longitudinal pressure wave is created that propagates outwardly along the vessel walls of the vasculature. Some solutions for measuring blood pressure attempt to make use of the velocity of this longitudinal pressure wave and the time it takes to travel between two arterial sites in the body of a subject.

However, this measurement is typically performed by recording the time interval between the passage of the arterial pulse wave at two consecutive sites, and therefore requires a sensor at each of the two sites. Achieving more accurate measurement requires the inclusion of further devices. In the pursuit of true ambulatory BP measurement, the provision of two or more sensors is inconvenient for the subject, and may again create false representations or cause discomfort in patients.

Recently, for ease of measurement, an electrocardiographic R or Q wave, detected from a chest lead of an ECG, has been combined with photoplethysmography (PPG), at a peripheral site such as a finger or ear lobe, for BP measurement. However, such a measurement has the problem of artefact. This is almost always due to interference with the PPG signal at the finger, but can also occur when chest wall movement disturbs the ECG leads. Such artefacts can usually be screened out if the signal is reviewed manually but, if automatic scoring is employed, then spurious interpretation can occur.

Hence, the current approach to measuring BP via PPG apparatus hinders true ambulatory measurements. Motion artefacts are often introduced that require manual review, which is cumbersome, time consuming and prone to error. In addition, increased respiratory effort can cause changes to the parameters required to accurately gauge BP, and these changes are also artefactual according to current approaches based upon PPG.

Other solutions for measuring BP do exist, such as the approach described in application WO 2018/189622. However, despite being less invasive and closer to an ambulatory measure, this approach described by this application still utilizes a cuff for measurement of blood pressure, albeit not in the oscillometric mode that is conventionally used. The use of a cuff can cause significant inconvenience to the user and can lead to increased anxiety resulting in a consequent elevation of BP.

In ‘Cuffless Blood Pressure Estimation Using Pressure Pulse Wave Signals’ (Liu at al, Sensors 2018, 18, 4227) a method of measuring blood pressure based on a pressure wave are described making use of a piezoelectric sensor that measures pressure directly in the same way as a BP cuff. However, this approach still suffers from the requirement that pressure calibration is required, adding an extra layer of complexity to the use of this solution as a true ambulatory monitor.

There exists a need to overcome the current aforementioned problems in the art.

### SUMMARY

According to an aspect of the invention, there is provided an ambulatory system for determining a at least one cardiac parameter at a fixed location of a cardiovascular system of a subject. The system comprises: a wearable sensor including an ultrasound transducer, wherein the wearable sensor can contact the skin of the subject and be positioned proximate to the fixed location; a data collection module that is in communication with the ultrasound transducer; wherein the ultrasound transducer is configured to detect a pressure wave passing through the fixed location, and wherein the data collection module is configured to collect data relating to the pressure wave passing through the fixed location, analyse the pressure wave, and determine at least one cardiac parameter based on the analysis.

The ultrasound transducer may comprise a piezoelectric ultrasound transducer. The ultrasound transducer may comprise a phased array imaging ultrasound transducer.

The data collection module may comprises a controller configured to apply the transform and determine the at least one cardiac parameter.

The controller may be remotely located from the sensor. The data collection module may further comprise a communications module connected to the ultrasound transducer. The communications module may be configured to transmit the collected data related to the pressure wave to the controller. The wearable sensor may include the communications module. The data collection module may comprise data storage.

The wearable sensor may comprise a patch for contacting the skin of the subject. The ultrasound transducer and at least part of the data collection module may be integrated into or integral with the patch.

The wearable sensor may comprise a removable module configured to connect to the patch when the patch is in contact with the skin. The removable module may comprise the ultrasound transducer and at least part of the data collection module. The removable module may comprise the ultrasound transducer and the communications module. The removable module may comprise the ultrasound transducer, the communications module, and data storage. The components within the wearable sensor may be connected by electrical connections. The removable module may comprise a waterproof housing. The housing may enclose the components of the wearable sensor. The removable module may comprise electrical contacts for connecting its electrical components to a power supply.

By removable, it is meant that the module may be separated, disconnected, or otherwise severed from the patch so that the patch and removable module can be used independently and individually while remaining combinable to form the system or part of the system. In other words, the physical, electrical, and acoustical connections between the patch and module when in their operational state are capable of being purposefully disconnected. The removable module may be therefore useable with a plurality of different patches. Vice versa, a single patch may be useable with a plurality of different modules. It is this interchangeability and the ability of a single module to be reused again and again by connecting and disconnecting from different patches that makes the device useful and sustainable in a field where waste is often extremely high. The patch and module may be considered as a multi-piece apparatus, as a kit of parts, or as individual components. The removability of the module also increases the ambulatory nature of the device—having a removable module enables storage and portability when not required, so that the user can effectively regulate when the device is used by connecting the removable module to the patch. A combined two-piece patch and removable module design also permits improvements to be made to each part independently, without having to entirely redesign the apparatus. This introduces a useful modularity and redundancy.

The patch may comprise a power source. The power source may be integrated within or integral with the patch. One or more power leads may extend from the power source for connection of the power source to another ultrasound transducer provided on a separate patch. Alternatively, one or more electrical contacts in communication with the power source may be configured to receive one or more power leads from another patch to transfer power therebetween. The system may comprise a second ultrasound sensor configured to be worn by a subject on the skin via a separate patch, and configured to connect to the one or more power leads to permit the second ultrasound sensor to be powered by the power source of the main patch. Alternatively, the system comprises a power source remotely located relative to the patch, the ultrasound sensor and/or data collection device are being powered by the remote power source. The remote power source may be provided in a power patch configured to be worn by the subject, the power patch and ultrasound patch connected by one or more power connections or leads. Accordingly, by providing separate battery systems, the ultrasound transducer may be powered for longer before replacing the base patch without compromising the ambulatory nature of the system. The power source may be disposed in the patch to sit between the skin and a housing of the transducer. The housing may incorporate a well or contour to sit around the power source.

The patch may comprise an adhesive layer for adhering the patch to the skin of the subject. The adhesive layer may comprise a biocompatible adhesive. The biocompatible adhesive may be a hydrocolloid adhesive.

The patch may comprise a contact layer. The contact layer may be suitable for contacting the skin of the subject. The contact layer may be suitable for improving ultrasound transmission between the ultrasound transducer and the skin of the subject.

The patch may be for location on the surface of the body of the subject. The patch may be a contoured patch that conforms to the anatomy of the subject. The fixed location may be the brachial artery. The patch may be configured for positioning on the skin of the subject in the region of the brachial artery. The ultrasound transducer may be located over the brachial artery. The ultrasound transducer may be configured for emitting and receiving ultrasound pulses to and from an artery, preferably the brachial artery, of a subject, through the subject's skin. The wearable sensor may be positioned in registry with an ultrasound echo window.

In some embodiments, the system can comprise further sensors or devices. For example, the system can comprise a second sensor, a third sensor, or a second and a third sensor. These devices can contact the skin of the subject, and are positioned proximate to a fixed location, for example a second and a third fixed location. Any of these devices may be comprised within a patch as described.

For example, in order to measure a central blood pressure as the cardiac parameter, the system may comprise a first device incorporating the wearable sensor and a second device incorporating another wearable sensor. The first device may be configured to detect a timing cue within the cardiac cycle of the subject, and the second device may be configured to detect a pulse pressure wave passing through the second fixed location. The data collection module may be configured to collect data relating to the transition of the pulse pressure wave passing through the second fixed location, thereby enabling determination of a pulse transit time (PTT) between the first and second fixed locations.

Any of the fixed locations may be part or all of body structures selected from one or more of: aortic arch, descending aorta, inferior vena cava, superior vena cava, brachial artery, femoral artery and carotid artery. In some embodiments, the first fixed location is comprised within the heart, optionally the aortic valve. In some embodiments, any of the devices are positioned in registry with an ultrasound echo window, which may be selected from one or more of: apical long axis, suprasternal, parasternal long axis left ventricle, parasternal short axis aortic Valve level, posterior at the height of the aortic arch, posterior immediately superior to the iliac bifurcation, carotid artery left, carotid artery right, subcostal four chamber short axis (lVC), Right supraclavicular (SVC), brachial artery left, brachial artery right, femoral artery left, and femoral artery right.

Performing the analysis on the pressure wave may comprise applying a transform to the pressure wave to obtain a calibrated pressure wave. Determining the at least one cardiac parameter based on the analysis may comprise determining a blood pressure from the calibrated pressure wave.

The pressure wave may comprise a pulse pressure wave (PPW). The pressure wave may comprise a flow velocity waveform. The pressure wave may be derived from motion changes in the wall of a blood vessel at the fixed location detected by the ultrasound transducer. The sensor may be configured to measure a diameter of an arterial wall, or an artery, and wherein the pressure wave is derived from the changes in the measured diameter. In some embodiments, the displacement or relative position of the arterial wall may be measured.

The at least one cardiac parameter may be selected from: systolic blood pressure; diastolic blood pressure; mean blood pressure; heart rate; heart rate variability; heart rhythm; peripheral blood pressure; or central blood pressure.

The ultrasound transducer may be configured to detect the pressure wave using M-mode ultrasound.

The system may comprise an actigraphy sensor configured to monitor the actigraphy of the subject and/or actigraphy events. The data collection module may be configured to store contemporaneous data from the actigraphy sensor and the wearable sensor together. The data collection module may be configured to perform one or more of the following steps: associate a timestamp with data from the actigraphy sensor and the wearable sensor, identify one or more actigraphy events in the data from the actigraphy sensor, identify data from the wearable sensor having a timestamp that is within a predetermined period before, at, and/or after the timestamp of the event, and store the event and the wearable sensor data together. The system may comprise one or more features of the actigraphy sensor and/or the actigraphy sensing system described below.

The system may comprise a display. The data collection module may be configured to determine at least one difference in time between consecutive peaks of the detected pressure waves, determine a heart rate based on the difference, and display the heart rate on the display. The heart rate may be displayed alongside or with the detected pressure waveforms.

According to an aspect of the invention, there is provided a non-invasive method for determining a cardiac parameter at a fixed location within the cardiovascular system of a subject. The method comprises: positioning a wearable sensor proximate to the fixed location, wherein the wearable sensor contacts the skin of the subject and the wearable sensor comprises an ultrasound transducer; detecting a pressure wave passing through the fixed location via the ultrasound transducer; collecting data relating to the pressure wave passing through the fixed location; analysing the pressure wave for the subject; determining at least one cardiac parameter based on the analysis.

Analysing the pressure wave may comprise applying a transform to the pressure wave to obtain a calibrated pressure wave. Determining at least on cardiac parameter based on the analysis may comprise determining a blood pressure from the calibrated pressure wave.

The method may comprise monitoring the pressure wave over a predetermined period. The method may comprise determining a time between adjacent peaks of the pressure wave, the time between peaks being indicative of a heart rate of the subject. The method may comprise calculating a variation in the time between adjacent peaks during the predetermined period.

According to another aspect of the invention, there is provided an ambulatory system for determining a cardiac parameter at a fixed location within the cardiovascular system of a subject. The system comprises: a wearable sensor including an ultrasound transducer, wherein the wearable sensor can contact the skin of the subject and be positioned proximate to the fixed location; a data collection module that is in communication with the ultrasound transducer; wherein the ultrasound transducer is configured to detect a pressure wave passing through the fixed location, and wherein the data collection module is configured to collect data relating to the pressure wave passing through the fixed location, analyse the pressure wave, and determine at least one cardiac parameter based on the analysis. The ultrasound transducer is connected to a power source. The system further comprises one or more power leads connected to the power source.

The power source may comprise a battery. The battery may be housed in the patch. The battery may be positioned to be beneath the module when received in the receptacle. The one or more power leads may be connected to the power source and configured to extend to another ultrasound transducer module in a remote patch on the subject's skin. The power source may be remote from the patch. The power source may be housed within a separate patch. The system may comprise one or more electrodes, the electrodes configured to be powered by the battery via the power leads. The electrodes may be configured for being adhered to a region of a patients chest. The system may comprise a temperature sensor and/or a respiratory rate sensor.

According to an embodiment of the invention, there is provided a system for facilitating diagnosis of cardiac rhythm and rate with the aid of a digital computer. The system comprises: an ambulatory blood pressure monitoring system and recording device; a processor and memory within which code for execution by the processor is stored. The processor comprises: an identification module configured to identify a plurality of P-P timing of a pulse-pressure wave (PPW); a calculation module configured to calculate a difference between recording times of successive pairs of the P-wave peaks and to determine a heart rate associated with each time difference; and a construction module configured to form an extended duration P-P interval plot over the set time period comprising each of the recording time differences and the associated heart rates; and a display operatively coupled to the processor, for displaying the extended duration P-P interval plot with a temporal point of reference in the extended duration P-P interval plot.

The system may display an ECG view produced at a traditional paper-based ECG recording speeds. The construction module may be configured to construct the extended duration P-P interval plot with a non-linear scale for the heart rates. The non-linear scale for the P-P rates may be displayed. The processor may comprise an identification or analysis module configured to: identify a potentially-actionable cardiac event within the P-P data; and select the plurality of PPW-wave peaks. The processor may comprise a diagnostic module configured to form a diagnosis based on PPW variability patterns in the extended duration P-P interval plot. The diagnostic module may be configured to detect atrial fibrillation by identifying a Gaussian-type distribution of PPW variability in the extended duration P-P interval plot.

According to another aspect of the invention, there is provided a method for facilitating diagnosis of cardiac rhythm disorders with the aid of a digital computer. The method comprises the steps of: receiving PPW data of a subject for a set period of time; determining one or more time differences between peaks in consecutive waves of the PPW data; determining a heart rate from the determined difference; and displaying the one or more heart rates on a display device. More specifically, the method may comprise one or more of the steps of: monitoring and recording cutaneous PPW of a patient; retrieving the cutaneous PPW data for a set time period and identifying a plurality of wave peaks; calculating a difference between recording times of successive pairs of the peaks and determining a heart rate associated with each time difference; forming an extended duration P-P interval plot over the set time period comprising each of the recording time differences and the associated heart rates; displaying the extended duration P-P interval plot and identifying a temporal point of reference in the extended duration P-P interval plot; and displaying at least part of the ECG data preceding and following the temporal point of reference as context in at least one accompanying ECG plot and or PPW waveforms.

The method may comprise the steps of: identifying a potentially-actionable cardiac event within the PPW data; and selecting the plurality of PPW peaks data prior to and after the potentially-actionable cardiac event. The method may comprise the step of forming a diagnosis based on PPW rate variability patterns identified in the extended duration P-P interval plot. The method may comprise the step of detecting atrial fibrillation by identifying a Gaussian-type distribution of PPW variability in the extended duration P-P interval plot. The method may comprise at least one of the steps of: including a background information plot with the extended duration P-P interval plot comprising one or more of activity amount, activity intensity, posture, syncope respiratory rate, blood pressure, oxygen saturation (SpO₂), blood carbon dioxide level (pCO₂), and temperature; and layering or keying background information with the extended duration P-P interval plot comprising one or more of activity amount, activity intensity, posture, syncope, respiratory rate, blood pressure, oxygen saturation (SpO₂), blood carbon dioxide level (pCO₂), and temperature.

According to another aspect of the invention, there is provided an ambulatory blood pressure monitoring system for determining a cardiac parameter at a fixed location within the cardiovascular system of a subject. The system comprises: a wearable sensor including an ultrasound transducer, wherein the wearable sensor can contact the skin of the subject and be positioned proximate to the fixed location, wherein the ultrasound transducer is configured to detect a pressure wave passing through the fixed location, an actigraphy sensor configured to monitor the actigraphy of the subject, and a processor configured to associate data from the actigraphy sensor with data from the wearable sensor.

The system may comprise one or more of the features described above. The system may comprise a sealed housing adapted to be removably secured into a non-conductive receptacle on patch. The actigraphy sensor may comprise an accelerometer. The accelerometer may comprise a 3-axis accelerometer. The actigraphy sensor may be operable to identify actigraphy events based on movement of the sensor and one or more actigraphy event criteria. The processor and/or actigraphy sensor may be configured to generate and generate an interrupt signal or flag upon identification of an actigraphy event. Upon generation of an interrupt signal or flag, the signal or flag may be associated with the data from the actigraphy sensor and wearable sensor. The interrupt signal or flag may cause a separate data file to be created. The interrupt signal or flag may differ depending upon the identified event. The processor may be configured to store data following actigraphy events in locations corresponding to the type of event and/or the type of interrupt signal or flag.

The actigraphy sensor may be configured to determine changes between two or more predetermined body positions. The actigraphy sensor may be configured to determine when a person moves between standing, sitting, and lying-down body positions.

The system may comprise a server centrally accessible over a data communications network. and configured to receive the data for secure storage. The server may be configured to analyse the data. The server may be configured to encrypt the data.

The processor may comprise a data retrieval module. The data retrieval module may be configured to retrieve one or more samples of the ultrasound signals. The ultrasound signals may be stored as pulse pressure waves (PPW). The data retrieval module may be configured to retrieve one or more samples of the PPWs. The data retrieval module may be configured to retrieve samples of the actigraphy events. The processor may comprise an evaluation module configured to identify an actigraphy event from the samples of the actigraphy event data based on a actigraphy event criteria. The processor may be configured to determine a time at which an actigraphy event was identified. The processor may be configured to store data from the actigraphy sensor and the ultrasound sensor with a timestamp. The processor may comprise a correlation module configured to identify samples of the ultrasound signal and/or PPW signal that were sensed at the same time as the actigraphy event. The processor may comprise an output module configured to output the actigraphy event and the samples of the ultrasound signal and/or PPW signal that were identified.

The processor may comprise an association module configured to combine the ultrasound signal or the PPW signal and the actigraphy event sensed at the same time into a single data track. The processor may be configured to identify ultrasound or PPW data having timestamps within a predetermined time period before and/or after the actigraphy event and store the identified data together with the actigraphy event. Ultrasound data from a predetermined time period prior to the actigraphy event and/or a predetermined time period after the actigraphy event may also be stored in a single data track.

The processor may be configured to determine an actigraphy event by an acceleration level exceeding an acceleration threshold. The threshold may be expressed based on recorded g-force. The processor may be configured to determine a fall based on a sudden peak in acceleration. The processor may be configured to determine periods of activity based on the actigraphy data.

The system may comprise a data retrieval module further configured to retrieve samples of physiology selected from the group comprising SpO₂, temperature, respiratory rate. The system may comprise temperature sensors, respiratory rate sensors, and/or SpO₂ sensors.

According to another aspect of the invention, there is provided an ambulatory system for determining a cardiac parameter at a fixed location within the cardiovascular system of a subject. The system comprises a wearable patch for contacting the skin of the subject, and a removable module configured to physically connect to the patch and to be held against the skin of the subject by the patch. The patch comprises an adhesive layer, an ultrasound transmission layer and a power cell. The adhesive and ultrasound transmission layers are configured to be in contact with the skin. The adhesive layer attaches the patch to the skin of the subject. The ultrasound transmission layer is configured to interface between the skin and an ultrasound transducer. The removable module comprises an ultrasound sensors comprising one more ultrasound transducers. The ultrasound transducer is configured within the module to contact the ultrasound transmission layer when the module is mounted to the patch. The ultrasound transducer is configured to detect a pressure wave passing through a vessel of the subject. The removable module and/or patch comprise a connection mechanism for attaching the removeable module to the patch. The removeable module comprises one or more electrical contacts configured to connect the ultrasound sensor to the power source in the patch. The removeable module further comprises a data collection system. The data collection system is configured to receive data from the ultrasound transducer and to store, communicate, and/or analyse the data to determine one or more cardiac parameters.

According to another aspect of the invention there is provided a wearable device for monitoring actigraphy and cardiac parameters of a subject. The device comprises one or more heart-rate sensors configured to monitor a pulse pressure wave at a fixed location of the subject. The device further comprises one or more actigraphy sensors configured to monitor changes in the subject's body position. The device comprises an adhesive for adhering the device to the subject's skin. The device comprises a controller configured to receive data from the heart-rate sensor and the actigraphy sensor and to correlate the data to identify trends and events.

According to another aspect of the invention there is a system comprising one or more ambulatory sensors each configured to determine arterial wall pulse pressure wave data of a subject, one or more processors configured to determine heart rate data from the arterial wall pulse pressure wave data, and one or more display devices configured to display the determined heart rate data to enable a physician to identify patterns in the data that may indicate heart arrythmias or other conditions.

There may be provide a method for determining one or more heart conditions, comprising determining pulse pressure wave data based on arterial wall measurements, determining a time difference between peaks of each pulse pressure wave, determining a heart rate based on the time difference, and displaying the heart rate on a display. The method may comprise enabling a zoom function to permit a physician to change the resolution of the data.

According to another aspect, there is provided a wearable sensor for determining a cardiac parameter at a fixed location within the cardiovascular system of a subject. The sensor is positionable proximate to the fixed location and comprises: a patch for contacting the skin of the subject, the patch comprising a power source integrated within the patch; and a removable module configured to connect to the patch when the patch is in contact with the skin, the removable module comprising a piezoelectric ultrasound transducer configured to detect a pressure wave passing through the fixed location. The removable module may comprise an actigraphy sensor configured to monitor actigraphy events.

According to another aspect of the invention, there is provided a data collection module configured to cooperate with an ambulatory blood pressure monitoring system having an ultrasound transducer and an actigraphy sensor. The module comprise: a communications module in communication with the ultrasound transducer and actigraphy sensor and configured to receive data from the ultrasound transducer relating to pulse pressure waves passing through a fixed location within the cardiovascular system of a subject from the transducer and to receive data from the actigraphy sensor relating to actigraphy events; data storage configured to store the received data; and a controller configured to: receive the data relating to the pressure wave and actigraphy events from the communications module; combine contemporaneous data from the actigraphy sensor and ultrasound transducer; and store the data in the data storage for analysis. Additionally or alternatively, the communications module may store the data received directly in the data store, and the controller may access the stored data. Additionally or alternatively, the controller may be configured to access the actigraphy and transducer data and perform analysis to identify one or more cardiac parameter based on the actigraphy events.

According to another aspect of the invention, there is provided a method of treating a patient suffering from hypertension. The method comprises monitoring a peripheral blood pressure at a fixed location within the cardiovascular system of the patient using the system described above; and administering an anti-hypertensive medication to the patient. The anti-hypertensive medication may be administered during periods of acute hypertension. The anti-hypertensive medication may administered during periods of chronic hypertension. The anti-hypertensive medication may be administered in a therapeutically effective amount. There may be provide a method of treating one or more of the conditions identified above, including monitoring the conditions and administering an appropriate medication effective in treating the condition.

The cardiac parameter in any of the aspects above is preferably a peripheral blood pressure. Alternatively, or additionally, the cardiac parameter may be a central blood pressure. In some embodiments, an ultrasound transducer may be configured to determine peripheral blood pressure in one mode using M-mode ultrasound, and to provide data for the determination of central blood pressure in another mode using doppler ultrasound or M-mode ultrasound wherein the device is in communication with another device that is also configured to provide data for determining central blood pressure, so that a pulse transit time may be determined between the two devices.

It is contemplated that any of the above features may be used in combination with each other, except where otherwise specified.

### DRAWINGS

The invention is further illustrated in the accompanying drawings.

FIG. 1 shows a schematic view of the underside (skin contacting side) of a patch for continuously recording the blood pressure of a subject according to one or more embodiments of the present invention.

FIG. 2 shows a schematic view of the side of another patch according to a further embodiment of the present invention.

FIG. 3A shows an exemplary uncalibrated pressure pulse wave signal.

FIG. 3B shows an exemplary calibrated pressure pulse wave signal.

FIG. 4 shows a schematic of a system according to some embodiments of the invention, wherein one or more patches are positioned on the body of a subject.

FIG. 5 shows a schematic of a system according to some embodiments of the invention, wherein information gathered from a subject is recorded and can be uploaded to a cloud system.

FIG. 6 shows a flow chart indicating a method for displaying heart rate of a subject based on pressure waveforms.

FIG. 7 shows an exemplary heart rate chart resulting from the method shown in FIG. 6.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs.

Prior to setting forth the invention, a number of definitions are provided that will assist in the understanding of the invention.

As used in this description, the singular forms “a,” “an,” and “the” include plural referents unless the context clearly dictates otherwise. Thus, for example, the term “a sensor” is intended to mean a single sensor or more than one sensor or to an array of sensors. For the purposes of this specification, terms such as “forward,” “rearward,” “front,” “back,” “right,” “left,” “upwardly,” “downwardly,” and the like are words of convenience and are not to be construed as limiting terms. Additionally, any reference referred to as being “incorporated herein” is to be understood as being incorporated in its entirety.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The term ‘ambulatory’ as used herein means that the devices and or systems described herein are in some cases designed to be used by ambulatory patients, that is, patients who are mobile, and able to walk or otherwise move around. This means that the devices are portable, and can be used outside the clinic, without the need for constant connection to bulky external power sources or other equipment. In other words, the patient or subject is able to move and operate in normal life, outside of the small radius permitted by conventional cuff-based systems. The subject may be an outpatient. The term ‘ambulatory’ and ambulatory devices or systems particularly include wireless systems that do not require connection to a system that is not worn on the subject's body. Moreover, ambulatory systems are lightweight so as not to interfere with day-to-day activities of the subject. For example, a device that has a heavy battery pack or a dialysis machine that is connected to the subject but can be wheeled around cannot be considered to be truly ambulatory because it hinders the actions of that person and prevents, to a certain extent, them from leading an entirely normal life. The term ambulatory may be referred to as truly ambulatory or fully ambulatory.

The term ‘wearable’ is intended to mean that the object described as wearable can be attached to and worn by a subject. Wearable devices are affixed to a part of the subject and move with movement of the subject. Care should be taken not to confuse the terms wearable and ambulatory in this application and in general. Wearable devices are mountable to a subject. The term implies the ability for a device to be worn by a subject, but is unconcerned with the ability for movement of the subject as ambulatory does. The two terms may be combined to provide a wearable and ambulatory object.

The term ‘ultrasound transducer’ refers to a device which can produce/transmit and receive ultrasonic waves, and can be used in ultrasonic scanning applications by interpreting reflected signals from a target. The term is intended to be synonymous with the terms ‘ultrasound transceiver’, ‘ultrasound sensor’ and ‘ultrasound probe’. The parts of the transducer which act as the transmitter and receiver may be separate or combined. Various frequencies of ultrasound can be used, depending on the depth of penetration required. The choice of ultrasound settings used may therefore depend on the location monitored by the transducer. For example, a 15-35 MHz transducer can be used, however, at least for monitoring of the brachial, carotid, and/or femoral arteries using pulse wave Doppler scanning techniques, frequencies of at least 0.5 MHz, suitably at least 1 MHz can be used. An advantage of using lower frequencies includes a reduction in power usage, which can prolong the life of the device and reduce the need for bulky power supplies. As will be discussed below, ultrasound transducers may be operated in one of a plurality of modes to provide different pulse patterns and to obtain different resolutions and measurement speeds.

The term ‘ultrasound window’ as used herein refers to an area on the body surface which allows effective ultrasound imaging of the underlying to be achieved. If an ultrasound transducer is placed ‘in registry with’ (that is, positioned close to and possessing a line of view that corresponds with the respective ultrasound echo window) such an ultrasound window, this can allow scanning of particular body structures.

The term ‘pressure wave form’ or ‘pulse wave form’ as used herein refers to a measurement of pressure, or a surrogate for a pressure measurement, over time in a particular blood vessel. The blood pressure inside any given blood vessel varies over the course of the cardiac cycle, in particular in the aorta and arteries, due to their function in carrying pressurised blood from the heart. In general, an arterial pressure wave form will have a peak corresponding to the high pressure of systole (heart contraction) and a trough corresponding to the lower pressure of diastole (heart relaxation and refilling).

The term “pulse pressure wave” or “PPW”, which may also be referred to as waveforms and particularly arterial pressure waveforms, refers herein to a pressure wave of each heartbeat measured at suitable locations that have been pre-determined by the operator of the systems and apparatus described herein, for example at the brachial artery, or at other arteries such as the carotid or femoral artery. These locations can be referred to as ‘fixed locations’, although the precise location that is monitored may be dependent on the placement of the devices of the invention. For example, where the brachial artery is monitored, the location used for the detection of the PPW will be the portion of this vessel which is most effectively monitored by a device of the invention which is placed on the subject proximate to this location. It will be appreciated that the term ‘fixed’ refers to the choice of the operator to pre-determine the anatomical location or point where the sensors are positioned on the subject. The PPW is generally created during contraction of the heart, and is typically a longitudinal pressure wave produced by the left ventricle's contraction. The longitudinal wave propagates outwardly along the vessel walls of the vasculature. The pulse pressure wave may also be observed in the blood flow within an artery. Where PPW is referred to herein, the measured wave is from the vasculature itself, and particularly the walls of the artery being monitored, unless specifically indicated. This propagation through the vasculature causes deformations or oscillations of the arterial wall and can be directly measured, non-invasively to obtain a pressure waveform. The PPW may also be the product of superposition of the longitudinal wave (a forward wave) and its reflection from peripheral vessels.

The term ‘arterial stiffness’ refers to the degree of elasticity found in an individual's arteries. Increasing arterial stiffness may occur as a result of aging and atherosclerosis, and is associated with risk of cardiovascular events.

The terms ‘power supply’ and ‘power cell’ can refer to any suitable means of supplying power to one or more electrical or electronic components such as ultrasonic transducers and data collection modules. Suitable power supplies may include for example, cells, batteries including lithium-ion batteries, and the like.

The term ‘data collection module’ as used herein refers to any suitable means for collating, processing and/or storing data collected by the sensors of the invention The data collection module 50 may comprise a processor and data storage means, such as a flash memory. The data collection module 50 communicates with and collects the data from the sensors comprised in the devices of the invention, for example the ultrasound transducer.

The term ‘subject’ as used herein refers to a human or animal to which the invention is applied. Typically the subject may be a human where blood pressure monitoring over time is desired. Various of the embodiments of the invention as described herein may be useful for application to humans as subjects, but also could be of use when applied to animals. Veterinary uses could include the monitoring of livestock, pets and other domestic animals, racehorses, show animals, animal being used in pharmaceutical and similar trials, and so on. Clearly, this will require significant amendments to be made with regards to calculations, which would vary depending on the target animal.

The term ‘actigraphy’ as used herein refers to non-invasive monitoring of a subject's movement and rest activity. Actigraphy is generally considered to involve the identification of body positions of the subject, such as when the subject is sitting, standing, or lying down, and the transitions between them. The monitoring of a subject may extend to actions performed during each position, such as movement of the subject during sleep phases, and to monitoring of extreme events during the subject's activity such as sudden changes in body position due to a fall.

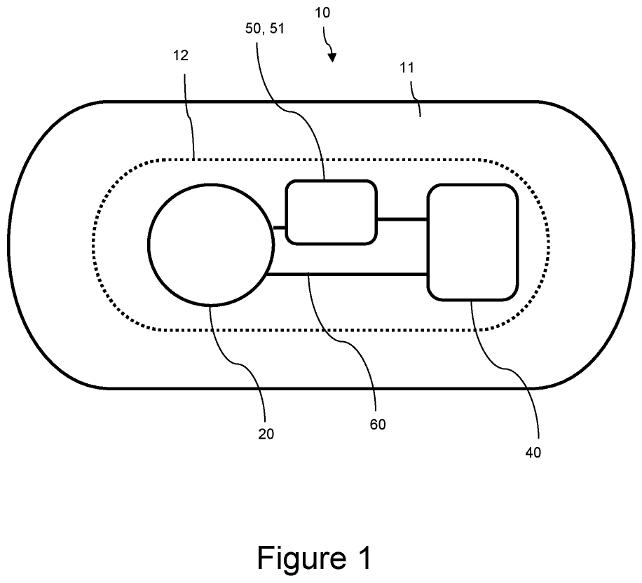

FIG. 1 shows a first embodiment of the invention, in which a device 10 comprises an adhesive patch 11 which allows the device to be applied to the skin of a subject. The patch comprises a number of components which are comprised within the area covered by the patch, thereby being placed in close or direct contact with the skin, in order to perform their functions. According to this embodiment of the invention, the components comprise at least one power cell 20, an ultrasound transducer 40 and a data collection module 50.

The adhesive patch 11 adheres to the skin of the subject using hydrocolloid or equivalent biocompatible adhesive. Biocompatible adhesives are preferable so as not to cause irritation. Hydrocolloid is particularly useful as it provides an adhesive that is transparent and breathable. The adhesive patch 11 is preferably contoured and flexible, in order to conform to the shape of the subject. Contouring may include the patch having a specific shape to match the shape of the area on which it is to be applied. Contouring may also include adaptation of the patch for the specific position it is to be positioned in. For example, the patch may be configured to bend or stretch a particular way to ensure that it continues to remain adhered.

The patch 11 may be configured to be attached in a particular orientation along the superior-inferior (or cranial-caudal) axis of the body, that is, with one end closer to the head, and the other closer to the feet. The adhesive patch may be applied at a single site the subject, to measure pressure wavefronts in different blood vessels. In embodiments, the adhesive patch is applied to one arm of the subject at a site corresponding to the subject's brachial artery. In embodiments where multiple patches are utilised, as a so-called patch array, the pressure wavefront may be monitored from a plurality of positions. In some instances, the patch is placed and configured to measure pressure waveforms in one or more of the carotid, brachial, and femoral arteries.

Applied centrally on the patch 11 is a contact layer 12. The contact layer 12 is adhered to the patch 11 and is dimensioned to occupy a portion of the patch 11 such that a boundary area of the adhesive patch 11 is left uncovered for adhering to the subject's skin. In other words, on the side of the patch 11 configured to contact the skin of the subject, the contact layer 12 is adhered to the patch 11 and sits within a boundary area comprising adhesive. The boundary area and adhesive thereof substantially surround the contact layer 12. Thus, when the patch 11 is applied to the subject's skin, the patch is adhered to the skin at the boundary area, thereby holding the contact layer 12 against the skin. The contact layer 12 is held in contact with the skin by the boundary area with sufficient pressure on all sides due to the boundary area surrounding the contact layer 12.

The contact layer 12 is positioned to at least align with the ultrasound transducer 40 as positioned in the patch 11. The layer 12 is arranged on the underside of the patch 11 and so will be in contact with the subject's skin when the device 10 is applied thereto. Accordingly, the layer 12, in use, sits between the subject's skin and the patch 11, and acts as an impedance matching or transfer layer, thereby improving the transmission of the ultrasound generated by the ultrasound transducer 40 to the subject and the artery. In this embodiment, the layer 12 is formed of a silicon-based material having a thickness comparable to that of the patch 11. Other materials may be used, such as a water-based gel, as appropriate.

The power cell 20 provides an integral power supply. By integral, it is meant that the power cell is wholly comprised within the device/patch. In other embodiments, described later, the power cell 20 may be non-integral with the patch, but may be provided in a separate patch, thus still forming an ambulatory system. The power cell 20 may be a lithium cell or battery and may be contained within a holder or other appropriate mounting assembly that is electrical connection with the other components within the device.

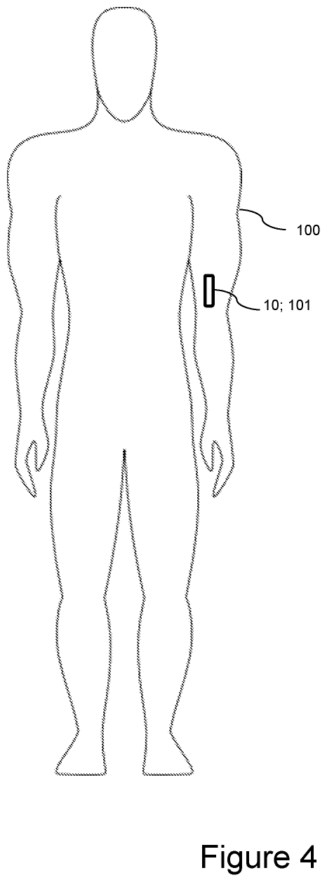

Suitably, the ultrasound transducer 40 is a piezoelectrical transducer. In one embodiment the transducer may be a phased-array ultrasonic imaging transducer. The ultrasound transducer 40 is able to both send and receive an ultrasound signal and so detect the arrival of a pulse wavefront in the brachial artery (or other appropriate blood vessel), through a suitable ultrasound echo window. Hence, the device of the invention is capable of directly measuring the progression of the pulse wavefront through a major blood vessel within the subject's body, for example, through the left or right brachial artery. For example, FIG. 4 illustrates a patch 10 or 101 provided on a subject 100 near the brachial artery. In one embodiment the device is able to determine the progress of the pulse wavefront directly by measuring the time taken for the pulse wavefront to progress across the field of the ultrasound echo window which incorporates the major vessel.

The ultrasound transducer is operable in one of a number of modes. As will be familiar to the skilled person, ultrasound transducers may operate in an A-mode, a B-mode, an M-mode and a Doppler mode, among others. As will be well understood by the skilled person, A-mode is used to scan a single line through tissue using a single transducer, while B-mode permits a plane within the body to be viewed, typically by making use of a transducer array. The M in M-mode stands for ‘motion’ and utilizes a rapid sequence of B-mode scans. The images obtained in M-mode can be sequenced to identify changes in the vasculature over a scanning period. Doppler mode measures and enables visualization of blood flow within the vasculature by making use of the Doppler effect.

In order to determine a pulse wavefront and/or a PPW in the walls of the artery, the ultrasound transducers 40 described herein utilize the M-mode. In some embodiments, transducers may also utilize other modes in addition to the M-mode, such as the Doppler mode to visualize blood flow. The comparatively high temporal and axial resolution makes M-mode ultrasound most useful in measurement of the pulse wave along vessel walls when compared to other forms of ultrasound measurement. The data collection module 50 may comprise a processor and data storage means, such as a flash memory. The data collection module 50 communicates with and collects data from the ultrasound transducer 40. Communication between the components 20, 40, 50 may occur via a wire, strip, ribbon or other suitable electrical connection. According to the device shown in FIG. 1, the electrical components 20, 40, 50 are connected by an electrical strip 60, which preferably is flexible in order to maintain connections between the components despite changes in position or movement of the subject. The electrical connections within the patch 11 may comprise a flexible circuit, configured to conform to the anatomy of the subject.

The data collection module 50 may simply act as a data store, as a wireless transmitter of data from the patch to a remote device, and/or may comprise a controller or processor that is capable of analysing data collected the ultrasound transducer 40. In the latter case the analysed data may also be stored within the data collection module or transmitted remotely.

The data collection module 50 may further comprise a Wi-Fi, 4G, and/or Bluetooth network-enabled sender/receiver module 51 to compare data with devices located elsewhere, either on the subject or to transmit data to a cloud based software platform (not shown). Other communication protocols may be employed to communicate data.

In the above embodiments, it is envisaged that the components 20, 40, 50, 51 of the patch 11 are integral with the patch 11 so that the entire device 10 can be applied to the subject's skin, and removed and discarded once the relevant data has been collected. In other embodiments, other arrangements of the device 10 may be utilised, as will now be discussed.

The data collection module 50 may send data from its sender/receiver module 51 to a controller elsewhere. The external and remotely located controller may be configured to analyse the data sent by the data collection module 50. In some embodiments, the data collection module 50 and controller may be considered to be part of a wider data collection system or module.

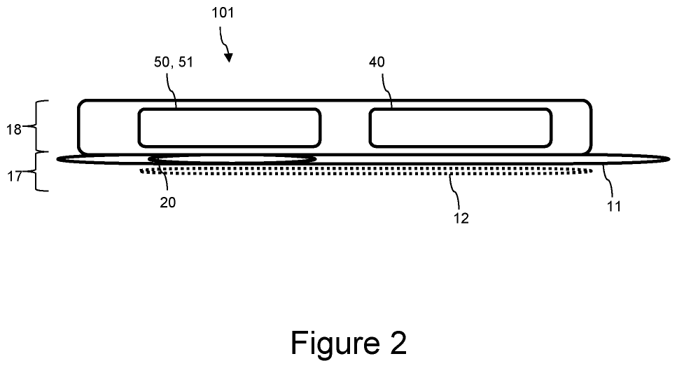

FIG. 2 shows a second embodiment of the invention, which comprises the features shown in FIG. 1 in an alternative arrangement. The device 101 comprises a single-use patch 17 and a re-usable, removable, ultrasound module 18. By re-usable and removable, it is meant that the module 18 may be attached to and severed from a plurality of different patches 17.

The single-use patch 17 includes the adhesive patch 11, the contact layer 12, and the power source 20. The power source 20 is integrated into the adhesive patch 11, and the contact layer 12 is provided on the underside of the patch 11 as described above. The formation of a composite patch 17 by combining these elements results in a flexible, lightweight, convenient patch to be applied to a subject's skin.

The patch 17 is compatible with at least one transducer measurement module, such as the removable ultrasound module 18 to enable measurement of relevant physiological parameters. The patch 17 may therefore incorporate one or more connection elements to connect with corresponding elements on an appropriate measurement module i.e. the transducer measurement module. The connection element may be a passive connection, configured for affixing the transducer measurement module onto the patch and maintaining the module in position so that the ultrasound transducer is correctly arranged relative to the artery that measurements are to be taken from. In other embodiments, the connection element may comprise contact points for electrical connection between the module and the power source 20. The connection element may comprise a non-conductive receptacle, securely adhered to a strip of the patch on the opposite surface to the contact layer.

To enable power to be supplied to the measurement module, the power source 20 may incorporate exposed contact points for electrical connection with corresponding contact points of the measurement module. These may be separate to the connection element, or, as described above, alternatively the connection elements may also comprise electrical contacts. In some embodiments, the connection elements are configured to connect the measurement module with the patch by sliding the measurement module across the upper surface of the patch 17, the upper surface being the surface that is exposed when the patch is applied to the subject's skin, before a connection is made.

The connection between the power source 20 and the contact points comprises electrical connections, typically in the form of a flexible circuit or flexible circuitry.

In this and other embodiments, the patch may comprise a laterally-extendable strain relief to prevent pressure being placed on the flexible circuitry. The strain relief is defined in the flexible circuit and formed to facilitate extension and rotation of the flexible circuit in response to tensile and torsional forces.

The ultrasound module 18 comprises the data collection module 50 in the form of a data store 52 and send/receive module 51 and the ultrasound transducer 40. As described above these components are operatively connected using appropriate electrical connections.

Preferably, the ultrasound module 18 comprises a housing surrounding the transducer 40 and data collection module 50. The housing may comprise connection elements corresponding to connection elements of the patch 17 for connection therewith. The housing may be entirely self-contained and the components enclosed therein so that the module 18 is waterproof and robust.

In some embodiments, the single-use patch 17 may incorporate a security device. The security device is configured to determine the identity of the module being connected to the patch. If the module is not recognized by the security device, the device will prevent power from being supplied to the module. The module may also include a similar security device configured to identify itself to the patch.

The combination of a re-usable module with a single use patch results in a cheaper system because the patches can be developed relatively cheaply and the expensive components found in the module are retained. The combination also permits different measurements to be performed depending on the desired outcome. A separable module needs to be calibrated far fewer times during a measurement cycle when compared to disposable patches incorporating all modules, as there is only one set of transducers to calibrate.

According to such embodiments the patch may be oriented and positioned appropriately in order to optimise the collection of sensor data.

In some embodiments, the patch 11 may be assembled from several layers including a structure/support material, an adhesive layer using hydrocolloid or equivalent biocompatible adhesive, a hydrogel component and an outer liner. The electrical strip 60 which connects the components may further comprise two layers of electrical circuit insulator to create an electrical circuit.

In one embodiment, the invention incorporates a configuration wherein a plurality of patches 11 are applied to the subject, and work in combination through coordination of their data modules 50. The plurality of patches 11 may be interconnected via a cable system, or via Wi-Fi, 4G or Bluetooth sender/receivers 51 and cooperate to generate sensor data necessary to measure and accurately determine real time parameters.

In some embodiments, the ultrasonic transducer 40 is positioned so as to monitor, via the appropriate ultrasound echo window, one or more blood vessels selected from: aortic arch; descending supraorbital artery, inverse facial artery, superficial temporal artery, maxillary artery, vertebral artery, aorta; inferior vena cava; superior vena cava; carotid arteries, brachial artery, radial artery, iliac arteries, subclavian artery, anterior tibial artery, posterior tibial artery, and femoral artery, or any combination of these locations. In a further embodiment of the invention (not shown) the device 10 may operate in combination with a separate ambulatory ECG monitoring system, such as a conventional Holter device, or another device 10 incorporating an integral ECG sensor. Hence, the patch 11 may communicate with and receive ECG data directly from the ECG monitoring system. Alternatively, the device may incorporate an integral ECG sensor. The ECG sensor may be integrated with the patch 11 or the module 18.

In one embodiment, there is provided a system comprising an ambulatory apparatus for applying to a subject, the apparatus comprising at least one patch which is applied to the subject on one or more parts of the body, and which remains in position for a period which may be of a duration of one or more hours, one or more days, or one or more weeks.

The apparatus acts to provide real-time monitoring of parameters associated with blood pressure, as is elaborated on below. These measurements may be made available to a user of the invention, such as the subject themselves, or a medical professional. As such, the apparatus may also comprise a display, which may be on an associated device for viewing by a user of the invention, or may transmit information via a wired or wireless system to a remote computer, to a remote or local storage device for later inspection, and/or to one or more so-called ‘smart’ device such as a telephone, laptop or tablet.

Such ambulatory apparatuses allow for blood pressure to be continually monitored under non-clinical conditions. This can allow instances of extreme blood pressure which might otherwise be asymptomatic to be detected, and the subject and/or a medical professional to be alerted. Similarly, blood pressure behaviour can be seen and/or recorded over long periods of time, allowing the detection of prolonged periods of abnormal levels, or trends of blood pressure readings over time.

This approach may be particularly useful when used to monitor the effect of particular treatments. Pharmaceutical and other treatments, for hypertensive or non-hypertensive conditions, may have effects on blood pressure, directly or indirectly, which may not be noticed at the time of a check-up in a clinical setting. As a result blood pressure can be viewed and/or recorded under various real-life conditions under particular circumstances, such as a change in a pharmaceutical strategy with a particular patient. This can allow outcomes like efficacy of hypertension treatments, or side effects on blood pressure of non-hypertension treatments to be measured, and can allow dosages to be revised in consequence.

A technical advantage is that the device of the invention is able to provide BP data in real-time via a minimal intervention approach to a medical sensing. This gives the subject the significant benefits of a comfortable, wearable device that does not inconvenience or interfere with their daily activities in order to gain a true representation of peripheral BP.

According to yet further embodiments of the device of the invention. additional sensors may be comprised within the one or more patches 11, or in separate patches or devices, including, but not limited to: an accelerometer; pulse detecting sensors such as photoplethysmographs or pulse oximeters; galvanic skin response sensor (sweat sensor); sensors that measure sweat composition including glucose, lactate, sodium and potassium content in sweat; and thermocouple or thermistor (temperature). The additional sensor(s) may communicate with the data collection module 50 and provide supplementary physiological data that may be prognostic or diagnostic in value. For instance, changes in these data may correlate with particular blood pressure values (or vice versa), thereby allowing improved accuracy in the detection of any episodes of abnormal blood pressure. Specific embodiments of the device incorporating one or more additional sensors are discussed in more detail below.

The invention provides, in one or more additional embodiments, at least one non-invasive method for determining peripheral BP in a subject, comprising determining a pulse pressure wave in a blood vessel located within the body of the subject via use of at least one ultrasound sensor applied to the skin of the subject. Suitably the ultrasound sensor comprises a piezoelectric ultrasound transducer, optionally a phased array imaging ultrasound transducer. In one embodiment of the invention the method is performed over a period of at least one hour, suitably at least two hours, at least six hours, at least 24 hours, at least 48 hours and not less than one week. In a further embodiment, the method is performed over a period of not less than one month, not less than six months, optionally for not less than one year.

In a specific exemplary embodiment of the invention, the entire system consists of two calibrated, standard automatic brachial blood pressure units that can measure right and left arm pressures simultaneously or separately via remote control. They are able to complete repeat readings and create BP averages and follow a pre-determined or programmable protocol to calibrate a combined sensor patch comprising a transmitter/receiver ultrasound array for the subject. The sensor patch may be connected to, or otherwise communicate with, a standard computer, or may be connected to a tablet-like or smartphone device for real-time monitoring and subject data input and calibration.

In one aspect, the device of the invention is a sensor patch that may comprise a contoured adhesive patch with an integral power supply (e.g. a lithium cell or battery) and appropriate ultrasound echo transducer for the location of the patch and the depth of field required. In one embodiment of the invention, the ultrasound transducer comprises a phased-array ultrasonic imaging transducer. The sensor patches may be connected to each other to facilitate communication of data and instructions, either via a cable system or via Bluetooth/Wi-Fi/4G and also to a recorder system. Each sensor patch may be specific to the location and contoured to fit that anatomy for the subject's comfort. The sensor patch is capable of monitoring, but not exclusive to and not limited to, all or any of the following standard ultrasound echo windows: apical long axis; suprasternal; parasternal long axis Left ventricle; parasternal short axis Aortic Valve level; posterior at the height of the aortic arch; posterior immediately superior to the iliac bifurcation; carotid artery Left; carotid artery right; subcostal four chamber short axis (IVC) right supraclavicular (SVC); brachial artery left; brachial artery right; femoral artery left; femoral artery right. In a preferred embodiment, the patch is optimized for placement on an ultrasound echo window proximate to the brachial artery left and/or brachial artery right, typically this is on the inner side of the elbow (see FIG. 4) In a further embodiment, the placement of the sensor patch can be on a temporal artery and can be used for determining the action of that artery for the purpose of predicting or monitoring characteristics of stroke or intracranial bleeding. In general, provided that a readily accessible ultrasound echo window exists that is proximate to an artery, the patch can be applied in order to monitor the pressure wave in the arterial walls.

In some embodiments, there may be provided an ambulatory blood pressure monitoring system utilising actigraphy monitoring techniques. The system is configured to non-invasively monitor the daily activity of the subject with whom the system is used and to whom the patch is applied. Actigraphy, as described above, is the monitoring of body position and body position transition of a subject in a non-invasive manner. Accordingly, the system incorporates one or more components within either the patch, in the case of the integral device or in the removable module, that connect to a processor to transfer data regarding the subject's activity. The advantage of such a system is that changes in blood flow volume can be directly correlated with changes in position of the subject, and so particular blood pressure readings can be performed using the device for different body positions. A system using actigraphy is especially useful in ensuring that a true resting blood pressure can be taken, as the actigraphy sensor is able to indicate when the subject is at rest and when they are not.

In general, the patch system utilising actigraphy comprises the patch and removable module described above in relation to FIG. 2, and is compatible with any other embodiments described herein.

The removable transducer module incorporates an actigraphy sensing system. The actigraphy sensing system is wholly housed within a sealed housing of the removable module. The actigraphy system is included within the electronic circuitry of the removable module that also includes the ultrasound transducer and processor. The actigraphy system is connected to the processor, at least, and draws power from the power cell that may be disposed in the patch or in the module.

The actigraphy sensing system comprises an actigraphy sensor. The actigraphy sensor, which may be an accelerometer or other motion sensor, electrically connects or interfaces with the processor.

The actigraphy sensor is configured to monitor the actigraphy of the subject. Data gathered by the actigraphy sensor is sent to the processor contained within the transducer module. The processor may analyse the data from the actigraphy sensor or associate it together with the ultrasound transducer data for subsequent analysis elsewhere.

Analysis of the actigraphy data comprises identification of specific actigraphy events within the sensed data. Actigraphy events, include: movement from one body position to another; sudden changes in body position; periods of rest; and periods of high activity. The processor identifies these events based on movement data and/or other gathered data from the actigraphy sensor.

Typically, movement of the subject is converted by the actigraphy sensor into one or more electrical signals. Where the actigraphy sensor comprises an accelerometer, the accelerometer may provide an electrical signal for each axis that it monitors. The accelerometer is typically a three-axis accelerometer.

Based on the movement data sensed by the actigraphy sensor, the processor is configured to identify actigraphy events. The actigraphy events may be identified based on predetermined actigraphy event criteria. The criteria may comprise, for example, an exceedance of a threshold value of acceleration in a particular axis, or an exceedance of a threshold averaged acceleration value over a time period, or identification of predetermined pattern of accelerations in the signal. The processor may also identify actigraphy events that appear anomalous.

The processor may be configured to label events using one or more flags, interrupt signals, or other metadata. The processor may also be configured to associate the actigraphy sensor data and the ultrasound data with timestamps. Data with like or similar timestamps may be associated and stored together. For example, upon identification of the event criteria, the processor may cause a flag or interrupt signal to be generated, the signal or flag indicating that an event has occurred. The signal timestamp may be compared with timestamps and data gathered from the ultrasound within a pre-determined time period before, at, and/or after the timestamp of the event may be stored as a data track or bundle relating to the event. Similar events may be stored together for subsequent analysis.

For example, where an actigraphy event is identified as the subject moving from a standing to a lying-down position, the processor generates a signal indicating the first timestamp of the change, and determines data falling within a predetermined time period after that first timestamp. The time period may, for example, be 10 minutes. The ultrasound data in that time period may subsequently stored with a label indicating that the subject was lying down, or stored in memory specifically related to data gathered when the subject was lying down. Accordingly, the transducer data can be categorised based on the actigraphy data to ensure that a true resting heart-rate and blood pressure measurement can be obtained based on resting data only, and with any other data removed.

In some embodiments of the system, there may be provided an ambulatory blood pressure monitoring system comprising a shared power source. Such a system may incorporate means for connecting a power source to two or more transducers, such as power leads extending from a patch-based battery and configured to connect to a separate patch without a power source. In doing so, the subject may be able to wear more than one transducer without requiring two power sources. In some embodiments, additional power beyond the capabilities of a single battery may be provided to ensure that the system can monitor for an extended period.

In a first embodiment, a patch is provided with a battery. The battery is configured to connect to a removable housing to power an ultrasound transducer therein via one or more electrical contacts. The battery may be disposed beneath the area to which the housing is mounted on the patch, and the housing may be contoured or shaped to fit around the battery. One or more power leads may extend from the battery away from the patch and separate to the housing, to connect to a further transducer elsewhere. This second transducer may be mounted on a separate patch on the subject, so that two different arteries may be monitored using a single power source.

In a different embodiment, a patch is provided on which the housing containing the ultrasound transducer is to be mounted without a power source. A power source is mounted within a housing to a separate patch. In this embodiment, the ultrasound transducer is powered by the power source on the separate patch and does not comprise an internal power source. The housing containing the power source does not comprise an ultrasound transducer. Therefore, the roles are separate, enabling a longer battery life, and for the subject to carry all the power required for extended monitoring with them, thus enabling enhanced ambulation.

The ultrasound transducers comprised within the sensor patch may monitor parameters such as pulsatile blood flow, vessel wall motion, blood volume and so forth, to gather data necessary to determine a pulse wave from the left ventricle as the blood passes through the brachial artery.

A pulse pressure wave (PPW) in an artery of a subject may be utilized to estimate peripheral BP of the subject (Liu et al. “Cuffless Blood Pressure Estimation Using Pressure Pulse Wave Signals”. Sensors 2018, 18, 4227). The PPW is a waveform produced by superposition of the forward wave created by the left ventricle's contraction and the reflection of the forward wave along the peripheral vessels. Subsequent analysis of a detected PPW can be utilized to measure BP.

During contraction of the heart, the longitudinal pressure wave that forms the PPW or at least part of it is created that propagates outwardly along the vessel walls of the vasculature. This propagation through the vasculature causes deformations or oscillations of the artery, and the arterial wall, and can be directly measured, non-invasively to create a pressure waveform. The ultrasound transducer is configured to measure changes in the diameter of structures, in this case walls of the artery being monitored. The changes in arterial wall diameter represent the passage of a pressure waveform, and it is this pressure waveform that corresponds to the action of the heart. By measuring the change in diameter of the walls of the artery, a representation of a pressure waveform is provided in millimeters (mm). Whilst uncalibrated, this waveform is directly proportional in morphology to that of a normal blood pressure curve as measured by an invasive fluid filled, or solid state catheter placed in the arterial system. The measured, uncalibrated pressure waveform can be calibrated by performing a single calibration routine with a standard cuff based device. Through this calibration routine, the uncalibrated waveform can effectively be mapped to the subject's blood pressure and cardiac activity and so can subsequently be used to accurately measure the subject's blood pressure and numerous other cardiac parameters. Analysis may be performed to determine parameters or indicators that are useful in performing diagnoses.

Ultrasound transducers are capable of non-invasively detecting and determining the PPW. Analysis of the PPW based on an ultrasound transducer measurement can be utilized to measure parameters including, but not limited to: heart rate; contractile force; peripheral blood pressure; central blood pressure; heart rhythm; heart rate variability. Based on the analysis, diagnoses, not exclusive to and not limited to, of the following conditions may be performed: cardiac rhythm disorders; normal sinus rhythm; sinus bradycardia; sinus tachycardia; premature atrial beats; premature ventricular beats; atrial fibrillation; atrial bigeminy; atrial trigeminy; atrial quatrigeminy; ventricular bigeminy; ventricular trigeminy; and ventricular quatrigeminy. Ultrasound transducers are capable of non-invasively detecting motion changes in the walls of blood vessels at their respective locations, and determining a pressure waveform from the motion changes.

The method used to determine BP based on a measured parameter using an ultrasound transducer may depend on the quality of the data available. For a noisy trace it may be most reliable to use a thresholding measurement set above the level of background noise, with the time of the pulse wave arrival set by the trace exceeding the threshold. If cleaner and more detailed data is available, features of the waveform can also be measured, and in such cases details such as the peak can be used as a marker for the pulse wave arrival. Waveform analysis may be automatic, such as if carried out by a computer, or may require human input, such as a medical professional. In some cases automatic analysis can be moderated by input from a human user and/or improved automatic algorithms.

In some embodiments, as an alternative to measuring and determining peripheral blood pressure, more than one device or sensor may be used to enable determination of central blood pressure. Suitable techniques for this are described in the application WO 2019/073236 A1. As described therein, a pair of sensor devices may be configured to determine the passage of a wave between two fixed locations in the cardiovascular system. A processor may be configured to determine a pulse transit time, which in turn enables the determination of a pulse wave velocity. From the pulse wave velocity, a central blood pressure may be determined. Using the devices described herein, the central blood pressure may also be determined based on a PPW measured at each of the fixed locations using m-mode ultrasound. In alternative methods, the sensors may use doppler flow ultrasound to determine a flow velocity. For central blood pressure, the two-piece patches may be applied at the brachial artery and carotid artery.

Calculation of Blood Pressure

In order to measure BP in each individual subject accurately via this non-invasive technique, the system may undergo a calibration step as part of the subject set up. The setup may comprise use of a standard digital brachial pressure cuff or other standard BP measurement device that provides standard measures of Systolic BP, diastolic BP, pulse pressure and heart rate. Once a standard calibration has been undertaken, a mathematical transformation function may be determined to be applied to data acquired from the sensor. In other words, the calibration is performed and a correlation identified between the PPW and parameters associated with blood pressure.

Best practice for calibration may include the standard technique of blood pressure measurement, with the subject resting for 5 minutes on a seated stool in a quiet room with their non-dominant arm being measured 3 times sequentially, with the average of 3 recordings being used as a baseline. It may be prudent to measure the subject in several positions to gain a better accuracy for the various activities that can be measured if an accelerometer is comprised within the sensor patch of the invention. By way of example, baseline measurements may be taken whilst the subject is sitting, standing and supine, with 3 recordings being used at each position and the average (mean) of each being used. It may also be prudent to measure both left and right arm simultaneously in the various positions.

A pre-determined calibration program may be followed, as set out below:
1. discuss procedure with subject and gain any necessary consent
2. place on at least one sensor patch, whilst subject is lying down
3. test device connection to peripheral blood pressure recorder
4. have subject lying down for 5 minutes
5. automatic BP recording and upload of averages to sensor patch to capture data with an accelerometer/body position data
6. subject to stand for 1 minute
7. automatic BP recording and upload of averages to sensor patch to capture data with an accelerometer/body position data
8. subject to sit in a stool with arms relaxed on tables by their side for 1 minute
9. automatic BP recording and upload of averages to sensor patch to capture data with an accelerometer/body position data
10. disconnect patient from calibration unit.

The above represents one particular calibration protocol and is no way limiting upon the methods or apparatus of the invention. Calibration estimates are, of course, more accurate with more pairs of blood pressure and PPW measurements. Further methods of perturbing blood pressure which can be used include Cold pressor (immersing the subject's hand or limb in cold water), physical exercise, mental arithmetic, sustained handgrip, controlled breathing, and pharmaceutical interventions such as nitroglycerin. These can lead to greater perturbations of blood pressure than postural changes alone and so improve calibration.

The device may further comprise an accelerometer connected to at least one other component, usually the data collection module. The accelerometer provides positional information on the body position of the subject during the calibration phase (lying, standing, seated, lying on their side etc). This may permit the use of different method of calibration or algorithms for performing the calibration. These methods and/or algorithms may be specific to a particular body position or set of body positions of the subject, thereby providing a more accurate representation of the blood pressure. For example, when the subject is lying down during night time rest, the columns of fluid in the body change and fluid pooling changes between standing upright/sitting and lying down. By calibrating the sensor for each of these individual positions, higher accuracy blood pressure measurements can be taken.

Furthermore, the number of measures taken during a 24 hour recording, for example, may be increased when compared to the current gold standard requirements of 14 day time and 7 night time measures. By increasing the number of measurements, a better risk prediction profile can be developed by measuring beat to beat blood pressure. This will greatly improve the accuracy of blood pressure measurements and give more detailed, individual blood pressure profiles to assist physicians on the application of hyper/hypotension management protocols, be it lifestyle modification, or pharmacotherapy- or device-based interventions.

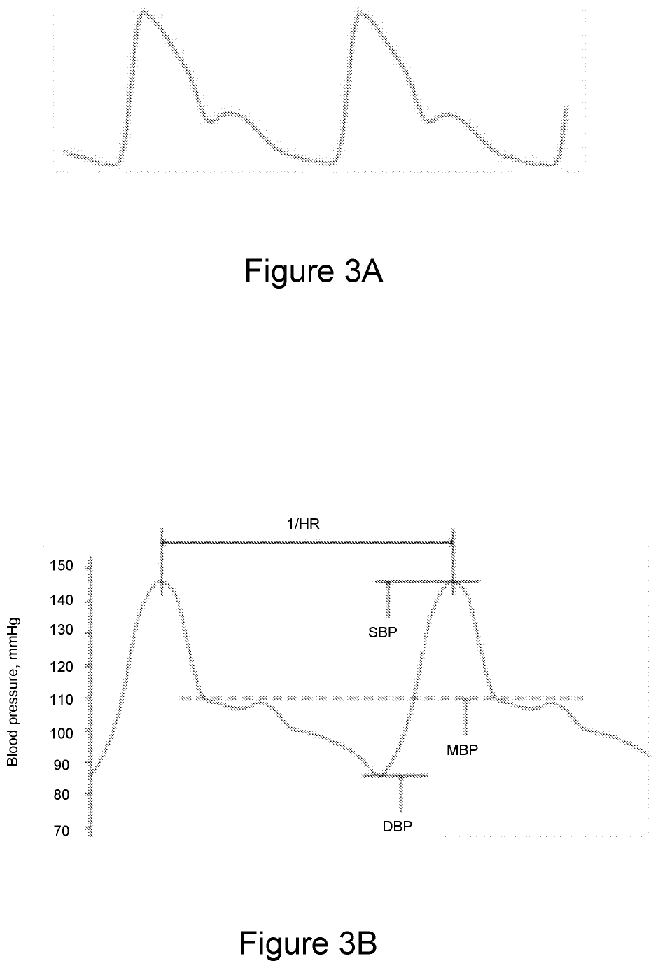

A typical uncalibrated PPW is illustrated in FIG. 3A. After the calibration has been performed and the mathematical function applied to the uncalibrated PPW, a calibrated PPW is achieved, an example of which is illustrated in FIG. 3B. In some embodiments, the mathematical function may be a transform or may alternatively be a modelling algorithm or other analysis that provides a pressure wave from which cardiac parameters can be determined.

As shown in FIG. 3B, the calibrated PPW provides a clear indication of several BP parameters. Measurement of the time between adjacent peaks of the calibrated PPW gives a measure of 1/HR (heart rate). The amplitude of the trough prior to the peak indicates diastolic blood pressure (DBP), while the amplitude of the peak is the systolic blood pressure (SBP). A mean blood pressure (MBP) can also be calculated between the SBP and DBP.

Monitoring the PPW over a period of time permits diagnosis of one or more of the following conditions: cardiac rhythm disorders; normal sinus rhythm; sinus bradycardia; sinus tachycardia; premature atrial beats; premature ventricular beats; atrial fibrillation; atrial bigeminy; atrial trigeminy; atrial quatrigeminy; ventricular bigeminy; ventricular trigeminy; and ventricular quatrigeminy. In particular, monitoring the time between the pulse beats in the brachial artery is particularly suitable for identifying and monitoring atrial fibrillation.

Methods of measuring blood pressure from PPW can also involve relatively detailed analysis of the pressure waveform itself. This can allow more information to be obtained, but does require an accurate picture of the pressure waveform to be available.

Accordingly, a method is provided for determining at least one cardiac parameter and/or peripheral blood pressure. In the method, a pressure waveform, typically a PPW, is detected using the ultrasound transducer in the device The ultrasound transducer measures and detects changes in the diameter of the arterial wall as the arterial walls oscillate due to the pressure wave that originated from the heart's motion passing through them. Typically, the sensor that comprises the ultrasound transducer is positioned at an ultrasound registry, suitably the brachial artery. Data received from the ultrasound transducer is passed to the data collection module. If the data collection module comprises a controller, the data collection module may analyse the pressure wave. If the data collection module comprises data storage and a communications module or just a communications module, the data collection module communicates the data to a remotely located controller for analysis. The remotely located controller performs an analysis on the received data. Analysing the pressure wave may comprise calibrating the pressure wave by applying a transform or mathematical function to it as described above. The outcome is a calibrated waveform that corresponds to the subject's blood pressure and can be subsequently utilised to determine peripheral blood pressure and/or other cardiac parameters.

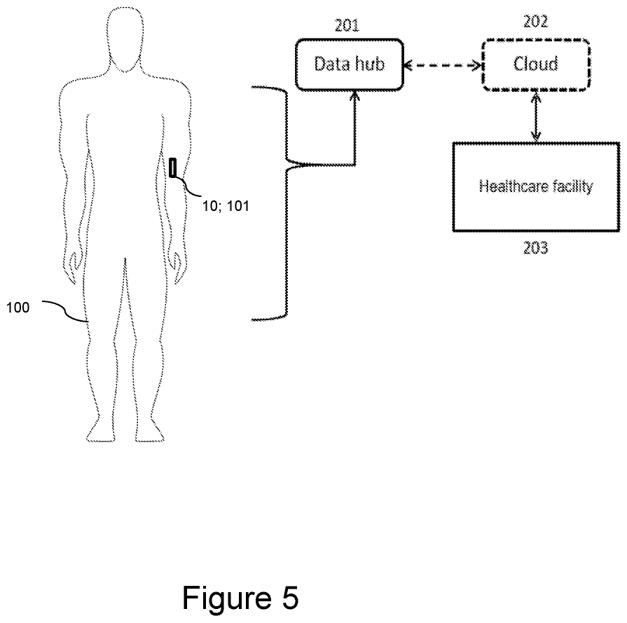

Algorithms which act to calculate blood pressure from pressure data gathered by ultrasound can be developed centrally and applied to the data generated by the invention. For example, patients undergoing cardiac catheterisation (specifically left heart cardiac catheterisation) may have fitted internal catheters which enable PPW data, and central or peripheral pressure to be measured directly, albeit in a clinical setting. Such patients could also have ultrasound data simultaneously gathered with devices or systems according to the present invention. The data generated by the catheters could then be used to determine the features of the concurrent ultrasound trace which relate to features such as the arrival of the pulse wave. Combining the internally measured, central measurements with the data gathered by the applied patches, will allow for a better baseline to which independently gathered patch data can be compared. This baseline can be continually updated as further data is collected. An example of this kind of system can be seen in FIG. 5, where data gathered from a healthcare facility 203, is uploaded to a cloud based service 202, and the developed algorithms used to determine features of ultrasound traces gathered by ambulatory systems according to the invention. Oversight can be maintained which allows for the disposal of spurious information.

Given that the invention can allow for the prolonged and continuous recording of hundreds of heartbeats, and associated blood pressure calculations, calibration can continue over time for each subject, so that the model used to calculate blood pressure can be updated. In addition, data from multiple subjects can be pooled so that the impact of other contributory factors can be taken into account, for example sex, ethnicity, body mass index (BMI), smoking status, and so on. These data can feed into a computer model developed over time with multiple subjects, in order to develop an enhanced, better calibrated model and mathematical function/transformation determination.

According to some embodiments of the invention, the ultrasound transducer may be configured to measure flow and characteristics of the flow within the artery. From the measurement of blood flow, a blood flow velocity can be measured. By identifying a flow velocity and a flow velocity waveform, analysis can be performed to convert the flow velocity waveform to a blood pressure measurement.

Ultrasound methods of imaging blood vessels, and particularly methods of measuring blood flow in said vessels, may make use of the Doppler effect (Kisslo J A and Adams D B “Principles of Doppler Echocardiography and the Doppler Examination #1”. London: Ciba-Geigy. 1987). Ultrasound-interacting objects (such as components of the blood) can move relative to the ultrasound emitter, to approach or recede, thereby causing a positive or negative Doppler shift in the received echo. Changes in this measurement can indicate a change in flow rate within the imaged vessel. Measurements made in this way can be used to determine the PPW.

In some embodiments of the invention an ultrasound transducer is located at the brachial artery, and in detecting by Doppler shift monitoring the change in blood flow caused by the heart, the onset of the pulse wave is determined. Methods to detect this can use continuous or pulsed ultrasound waves. While continuous waves can reliably measure relatively fast flow rates, they lack the ability to discriminate depth and therefore can be affected by noise from the whole tissue depth. Pulsed wave Doppler may therefore be of more use in the present context, since it can be tuned to detect data only from a certain depth.

Analysis of Device Output

PPW allows physicians to diagnose cardiac function by reviewing the timing between each PPW. Linking this to ambulatory blood pressure allows for the correct assumption when calculating pressure and clinical decision making for the correct treatment for a patient.

In order to allow physicians to review changes in rhythm abnormality based on the data, a further analysis is useful. Accordingly, the PPW data, and in particular the uncalibrated and calibrated waveforms, are used to produce central or peripheral arterial PPW to PPW interval data for presentation to a physician and for subsequent computerized analysis.

In particular, the further analysis described herein utilizes the distinctive peaks of the calibrated waveform to determine a heart rate and to subsequently display the heart rate to the physician to allow for temporal analysis of the patient's heart rhythm. Such analysis may be described as peak-to-peak or P-P and makes use of a P-P plot.

As described above, a single uncalibrated PPW waveform can be converted to a calibrated waveform in order to determine particular cardiac parameters. By making use of multiple consecutive waveforms, a heart rate can be determined and trends in heart rate monitored.

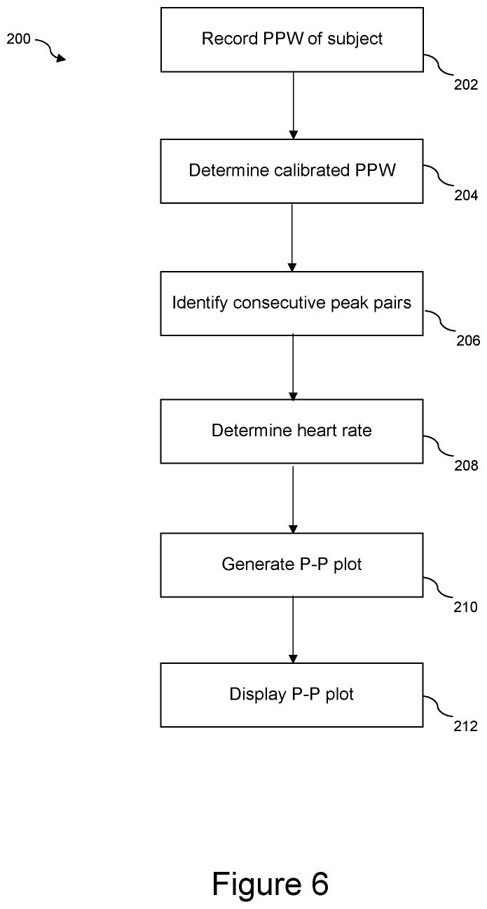

FIG. 6 shows a flow chart indicating a method 200 for this analysis and for displaying a heart rate on a digital display. At a first step 202, the PPW of a subject is recorded using the device described above. The cutaneous PPW of the subject is monitored and recorded using ultrasound transducer data. Based on the uncalibrated data directly from the transducer, at step 204, a calibrated PPW waveform is determined. From the calibrated waveform, a plurality of consecutive peaks in the waveform are identified at step 206. From each pair of peaks, at step 208, a heart rate is calculated based on the difference in time points at which the peaks were recorded. In other words, the time elapsed between peaks is used to give an inverse of the heart rate. The heart rate between each successive PPW waveform can therefore be determined, and an overall picture of the heart rate can be developed.

The heart rate is determined for each of a plurality of pairs of peaks within the waveform over a specific period of time. For example, the period of time may be 1 minute, 10 minutes, or any other suitable period.

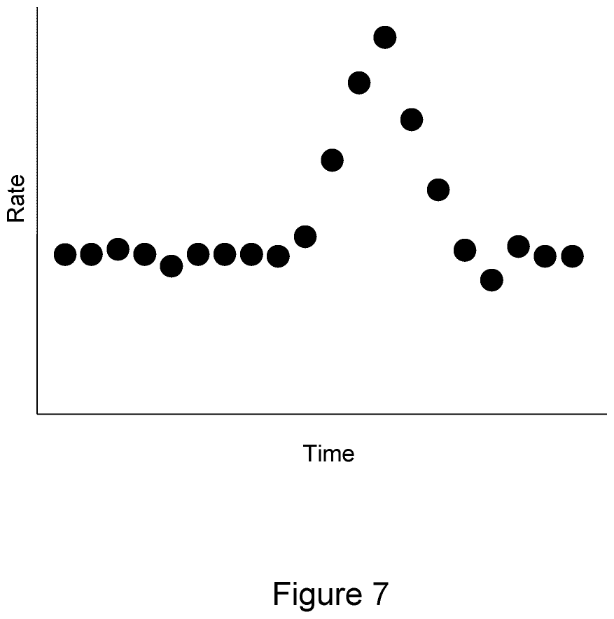

Based on each of the determined heart rates, at step 210, an extended duration P-P interval plot over the specific period is formed, indicating the time difference and the associated heart rate, so that a linear progression of heart rate over time can be plotted on a chart. The plot, an example of which is shown in FIG. 7, is displayed on an external monitoring device at step 212. A temporal reference point may be provided to indicate the time at which the measurements were taken. At least part of the PPW data preceding and following the temporal point of reference may be displayed as context in at least one accompanying PPW plot.

The data is presented in a format that includes views of relevant near field and far field PPW data, which together provide contextual information that improves diagnostic accuracy. The near field (or short duration) PPW data view provides a “pinpoint” classical view of a calculated pressure wave at traditional recording speed in a manner that is known to and widely embraced by physicians. The near field PPW data is coupled to a far field view that provides a lower resolution, pre- and post-event contextual view.

Both near field and far field PPW data views are temporally keyed to an extended duration P-P interval data view. In one embodiment, the P-P interval data view is scaled non-linearly to maximize the visual differentiation for frequently-occurring heart rate ranges, such that a single glance allows the physician to make a diagnosis. All two views are presented simultaneously, thereby allowing an interpreting physician to potentially diagnose rhythm and rate pre- and post-PPW collection and pressure calculation.

In other words, it is envisioned that P-P plots may be delivered on a non-linear y-axis scale, to aid in visualisation. In particular, the plots may be delivered on a logarithmic scale on the y-axis (i.e., a semi-log scale), typically a log-2 or log-10 scale. The non-linear scale may be applied on part or all of the axis.

Additionally, the x-axis representing time may be non-linear. In particular, periods of time which are considered of less interest, for example periods which have been assessed by automatic analysis or the input of a medical practitioner to represent sinus rhythm may be compressed or excised, such that periods of more interest can be seen. In this way, different episodes of particular rhythmic disturbances can be directly compared to one another.

It is particularly envisioned that the time period to which the beat-to-beat display plot corresponds can vary. It should especially be possible for a user of the invention to refer to a particular display plot and then ‘zoom in’ on a particular time period to see it in more detail. In this way points of particular interest can be examined. Similarly, the user could ‘zoom out’ to see a longer time period, and thereby gain an overview of heart function over a greater period of time.

In this way, the display plots produced are intended to be interactive, such that the pertinent information may be selected with ease by a user of the invention, such as a medical professional, or a user of a fitness-monitoring system. In comparison to a system which would show a static snapshot of cardiac data, it is particularly advantageous to select, rapidly and straightforwardly, different time periods within what may be a recording of long duration, so that for example areas of interest can be rapidly identified in a display corresponding to a long time period, and those areas can be more closely studied in a display of a shorter time period. Additionally, due to the difference in utility in different contexts of larger scale ‘macro’ level data represented by the beat-to-beat display plot, and smaller scale ‘micro’ level data, the ability to choose the nature of the displayed information allows efficient availability of the needed data at any time. These aspects enhance the presentation of diagnostically relevant PPW interval data, reduce time and effort needed to gather relevant information by a clinician and provide the clinician with an additional diagnostic tool, which is critical to accurate arrhythmia and rate diagnoses.

Additional data may be provided in addition to the PPW, P-P interval, or BP plots. Where the device incorporates an ECG sensor, the ECG data preceding and following the temporal point of reference may also be displayed as context. Upon introduction of an actigraphy sensing system, the sensed body position may be determined and displayed.

In addition to the display of the data, a processing system may be provided configured to analyse the P-P plot data and heart rate values derived from it. The processing system may be configured to identifying a potentially-actionable cardiac event within the PPW data. Based on the event identified, the processing system may select the plurality of PPW peaks data prior to and after the potentially-actionable cardiac event.

Other data that may be gathered and/or displayed alongside the P-P plot data includes one or more of the following: activity amount; activity intensity; posture; syncope respiratory rate; blood pressure; oxygen saturation (SpO₂); blood carbon dioxide level (pCO₂); and temperature. The data may be layered with the P-P plot to permit correlation of the data or for the P-P plot to be viewed with a particular context. By activity amount and activity intensity, it is intended to mean that parameters such as acceleration of one or more accelerometers is gathered and/or other actigraphy data as described above, and correlated to specific exercises or activities. Intensity may be determined based on the accelerations and the heart-rate during the activity, while amount may be based on the acceleration, the heart-rate, and/or the time period over which the activity was performed.

Following the analysis to determine the P-P data and plots, and subsequent display, a computer-implemented analysis of the data may be performed to identify trends and/or pattern in the data. Conventional statistical analysis and pattern identification techniques may be utilized to fit trendlines, identify anomalous readings, and to identify any data that may be worth investigation. The analysis may be performed using machine-learning techniques and neural networks that have been trained on existing data to recognize data that indicates specific cardiac events. If trends or patterns or anomalies are identified, the system may create a flag or note to be displayed alongside the data or at the point that has been identified in the data.

Examples of such analysis comprise determining variability in the P-P data. Using a moving average or other averaging, the variability of the heart rate in specific windows may be determined. The variability may be plotted separately and patterns identified in the data. Another example is the generation of distributions of the data based on a specific time period of the data. From the distribution, different distribution types may be identified, such as Gaussian or normal.

Further analysis may be performed to achieve computer-implemented diagnosis.

Certain arrhythmias may be relatively easily distinguished from a P-P (i.e. beat-to-beat) plot due to their characteristic effects on the heart rate. In this way, parts of a beat-to-beat plot may be subjected to rhythm analysis in order to be categorised as belonging to one or more ‘rhythmic categories’. The rhythm analysis will generally be an automated process, although in some aspects it may be possible to perform such analysis manually. Some non-limiting examples of these categories are described in greater detail below. Devices such the device described above can be used as a diagnostic tool, due to macro level pattern recognition possible through the provision of beat to beat plots. It has been shown that cycle length alone (that is, the duration from one measured variable to another such as the interval between ventricular depolarisations) can be used to diagnose and monitor an arrhythmia by applying standard mathematical indices of mean, mode, standard deviation, co-efficient of variance, and so on. These mathematical variables, combined with the visual macro level pattern recognition, are therefore able to be highly diagnostic and/or predictive of various types of arrhythmia.

Sinus rhythm is the normal functioning of a healthy heart, where the trigger for cardiac muscle contraction originates in the sinoatrial node and spreads through the heart. This leads to a regular contraction of the heart muscle. Sinus arrhythmia is where the interval between heartbeats varies, despite the trigger still occurring in the sinoatrial node. This can have a number of causes, such as respiration, or exercise. While mostly seen in younger healthy people, and generally of little concern, sinus arrhythmia can be a signal of heart disease, especially when it appears in the elderly.

Atrial fibrillation is a condition which causes an irregular and often abnormally fast heart rate, caused when the impulse generated by the sinoatrial node is overwhelmed by abnormal impulses generated elsewhere, such as in the roots of the pulmonary veins. It is the most common heart rhythm disturbance, affecting around one million people in the UK, and increasing in prevalence in older people. Atrial fibrillation is a marker for higher stroke risk. Episodes of atrial fibrillation may be marked by symptoms including heart palpitations, fainting, lightheadedness, shortness of breath, or chest pain, but in many cases episodes do not cause symptoms. Atrial fibrillation can be seen in a P-P plot as a ‘cloud-like’ dispersed pattern of irregular P-P intervals and/or by detection of a Gaussian-type distribution of variability.

Atrial flutter is a condition which shows similar symptoms to atrial fibrillation and often occurs in the same patients. However, this condition is in some cases treated differently to atrial fibrillation, and as a result distinguishing between the two is important. Atrial flutter is characterised by rapid onset periods of an elevated heart rate, with a more regular rhythm than is usually found in atrial fibrillation. This condition can be determined from the appearance of characteristic ‘flutter waves’ or ‘F waves’, being a pattern of regular, rapid atrial waves at a regular rate of more than 200 per minute.

Bigeminy is a condition where there is a regular alternation of long and short heart beats, giving a regular pattern of grouped pairs of heart beats. This condition is usually caused by ectopic heartbeats, that is, where the electrical trigger for cardiac muscle contraction originates outside the sinoatrial node. Trigeminy is a similar condition where triplets of heartbeats are seen.

To enable the data analysis and presentation of data described above, both in relation to blood pressure and heart rate monitoring, a system may be provided. The system may be for facilitating diagnosis of cardiac rhythm and heart rate with the aid of a computer.

The system comprises an ambulatory blood pressure monitoring system, including one or more patches as described above. The monitoring system also includes a data collection device or server, either within the patch or in a separate device such as a smartphone or computer system. The data collection device, which may be referred to as a recording device, receives the data gathered by the ultrasound transducer module and stores it in memory.

A processor comprising an identification module is configured to retrieve the stored or received data and to perform the method described above, by identifying a plurality of peak timings of a pulse pressure wave. The processor comprises a calculation module configured to calculate the difference between consecutive peaks and their recording times, and to determine a heart rate from these. The processor further comprises a construction module to construct a P-P interval plot as described above. The processor communicates the plot from the construction module to a display device for display to a physician or subject. The display device may comprise a computer system or smartphone, and the display device may be integrated into the same device that received the data and/or that processed the data.

The display may comprise an e-ink display configured to display the heart rate or other data such as an ECG in a traditional paper-based manner, i.e, as if it were being printed.

The aforementioned embodiments are not intended to be limiting with respect to the scope of any claims, which may be filed on applications filed in the future and claiming convention priority from this application. It is contemplated by the inventors that various substitutions, alterations, and modifications may be made to the invention without departing from the spirit and scope of the invention as defined by the claims.

1. An ambulatory system for determining a cardiac parameter at a fixed location within the cardiovascular anatomy of a subject, the system comprising:
a wearable sensor including an ultrasound transducer, wherein the wearable sensor is configured to contact the skin of the subject and be positioned proximate to the fixed location;
a data collection module that is configured to be in communication with the ultrasound transducer;
wherein the ultrasound transducer is configured to detect a pressure wave passing through the fixed location, and wherein the data collection module is configured to collect data relating to the pressure wave passing through the fixed location, and to analyse the pressure wave, and to determine at least one cardiac parameter.

2. The system of claim 1, wherein the ultrasound transducer comprises a piezoelectric ultrasound transducer.

3. The system of claim 1, wherein the ultrasound transducer comprises a phased array imaging ultrasound transducer.

4. (canceled)

5. The system of claim 1, wherein the controller is remotely located from the sensor, and the data collection module further comprises a communications module connected to the ultrasound transducer that is configured to transmit the collected data related to the pressure wave to the controller.

6. The system of claim 1, wherein the data collection module comprises data storage.

7. The system of claim 1, wherein the wearable sensor comprises a patch for contacting the skin of the subject and wherein the ultrasound transducer and at least a part of the data collection module is integrated into the patch.

8. (canceled)

9. The system of claim 7, wherein the wearable sensor comprises a removable module configured to connect to the patch when the patch is in contact with the skin of the subject, the removable module comprising the ultrasound transducer and at least a part of the data collection module.

10. The system of claim 7, wherein the patch comprises a power source, the power source being integrated within the patch.

11. (canceled)

12. The system of claim 7, wherein the patch comprises an adhesive layer for adhering the patch to the skin of the subject, wherein the adhesive layer comprises a biocompatible adhesive.

13. (canceled)

14. The system of claim 7, wherein the patch comprises a contact layer for contacting the skin of the subject and to improve ultrasound transmission between the ultrasound transducer and the skin of the subject.

15. The system of claim 7, wherein the patch is a contoured patch that conforms to the anatomy of the subject.

16. The system of claim 1, wherein the fixed location is the brachial artery.

17. The system of claim 1, wherein performing the analysis on the pressure wave comprises applying a transform to the pressure wave to obtain a calibrated pressure wave, and wherein determining the at least one cardiac parameter comprises determining a blood pressure from the calibrated pressure wave.

18. The system of claim 1, wherein the pressure wave comprises a pulse pressure wave (PPW), the pulse pressure wave being derived from motion changes in the wall of a blood vessel at the fixed location detected by the ultrasound transducer.

19. The system of claim 1, wherein the sensor is configured to measure the diameter of an arterial wall, or artery, and wherein the pressure wave is derived from the changes in the measured diameter.

20. The system of claim 1, wherein the at least one cardiac parameter is selected from: systolic blood pressure; diastolic blood pressure; mean blood pressure; heart rate; heart rate variability; heart rhythm; peripheral blood pressure; or central blood pressure.

21. The system of claim 1, wherein the ultrasound transducer is configured to detect the pressure wave using M-mode ultrasound.

22. The system of claim 1, comprising an actigraphy sensor configured to monitor actigraphy events wherein the data collection module is configured to store contemporaneous data from the actigraphy sensor and the wearable sensor together.

23. (canceled)

24. (canceled)

25. The system of claim 1, comprising a display, the data collection module configured to determine at least one difference in time between consecutive peaks of the detected pressure waves, determine a heart rate based on the difference, and display the heart rate on the display, preferably with the pressure waveforms.

26. A wearable sensor for determining a cardiac parameter at a fixed location within the cardiovascular system of a subject, the sensor being positionable proximate to the fixed location and comprising:
a patch for contacting the skin of the subject, the patch comprising a power source integrated within the patch; and
a removable module configured to connect to the patch when the patch is in contact with the skin, the removable module comprising a piezoelectric ultrasound transducer configured to detect a pressure wave passing through the fixed location.

27-30. (canceled)
